(19) United States
(12) Patent Application Publication
(10) Pub. No.: US 2010/0136035 A1
(43) Pub. Date: Jun. 3, 2010

(54) METHOD FOR MODULATING ATHEROSCLEROSIS

(76) Inventors: Daniel S. Greenspan, Madison, WI (US); William J. Burlingham, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 12/623,233

(22) Filed: Nov. 20, 2009

Related U.S. Application Data
(60) Provisional application No. 61/116,768, filed on Nov. 21, 2008.

(51) Int. Cl. A61K 39/00; A61K 38/39; A61K 31/7088; G01N 33/53; C12Q 1/06; A61P 9/10

(52) U.S. Cl. 424/184.1; 514/12; 514/44 R; 435/7.1; 435/39

(57) ABSTRACT

Immunoreactivity to collagen V is associated with development of atherosclerosis. Methods disclosed herein for modulating atherosclerosis and reducing an effect thereof, involve inducing immunological tolerance to collagen V. Methods for evaluating a risk of an individual to develop atherosclerosis and methods for diagnosing atherosclerosis are disclosed herein.

### CROSS-REFERENCE TO RELATED APPLICATIONS

This application claims the benefit of U.S. Provisional Patent Application No. 61/116,768, filed Nov. 21, 2008, incorporated herein by reference as if set forth in its entirety.

### STATEMENT REGARDING FEDERALLY SPONSORED

This invention was made with United States government support awarded by the following agencies: NIH AR047746. The United States government has certain rights in this invention.

### BACKGROUND

Atherosclerosis is a chronic inflammatory disease characterized by flow-obstructing deposits on inner arterial linings. The deposits, or plaques, contain cellular waste products and substances such as cholesterol and calcium. The plaques can rupture and set in motion a cascade that culminates in blood clots associated with gangrene, heart attack, and stroke. Immune responses to autoantigens are important components of the pathogenesis of atherosclerosis as evident from proatherogenic adoptive transfer of CD4⁺ T cells from atherosclerotic donors and reduced atherosclerotic lesions in immunodeficient animals. Autoimmune response to local autoantigens such as oxidized low-density lipoprotein (oxLDL) and heat shock protein-60 (HSP 60) is involved in establishing and maintaining the disease (see, e.g., Sherer, Y., and Y. Shoenfeld, Nat. Clin. Pract. Rheumatol. 2:99-106 (2006), Hansson, The Anitschkov Lecture, Atherosclerosis (2007), doi:10.1016/j.atherosclerosis.2008.08.039). Anti-HSP 60/65 antibodies as well as HSP 60/65- and oxLDL-specific T cells are significant predictors of atherosclerotic lesion development. To the extent that proatherogenic autoantigens are known, immunomodulation directed to these autoantigens can treat or prevent the disease. Mucosal immunization against oxLDL and HSP 60/65 in animals has resulted in prevention and amelioration of atherosclerosis. A role for autoimmunity in this disease is further evidenced in that subjects having autoimmune diseases such as rheumatoid arthritis, systemic lupus erythematosus, and systemic sclerosis often have accelerated and severe atherosclerosis, resembling coronary artery disease (CAD).

An autoimmune response to the α1(V) chain of collagen V predisposes individuals to obliterative bronchiolitis (Burlingham et al., J. Clin. Invest. 117:3498-3506 (2007)), the leading cause of chronic rejection of transplanted lungs in humans. The α1(V) chains of α1(V)₂α2(V) heterotrimers are in the interiors of collagen V/collagen I fibrils and are inaccessible to the immune system under normal conditions. In the absence of sufficient numbers of α2(V) chains to form α1(V)₂α2(V) heterotrimers, α1(V) chains form α1(V)₃ homotrimers (Haralson et al., PNAS 77:5206-5210 (1980). These homotrimers are excluded from the interior of type I collagen fibrils (Chanut-Delalande et al., J. Biol. Chem. 276:24352-24359 (2001)) and are instead exposed to the immune system, potentially initiating an immune cascade leading to obliterative bronchiolitis. Oral administration of collagen V reduced delayed type hypersensitivity to alloantigens in rats (WO 2007/120947).

α1(V) chains are specifically up-regulated in atherosclerotic plaques (Ooshima, Science 213:666-668 (1981)). To date, collagen V-specific autoimmunity has not been associated with atherosclerosis.

### BRIEF SUMMARY

The present invention arises from the inventors' observation, disclosed herein for the first time, that human and non-human subjects having coronary artery disease, but not control subjects, exhibit antigen-specific cellular immunoreactivity to collagen V. From this observation, the inventors understand that incidence of atherosclerosis and its associated physiological symptoms can be modulated by inducing immunological tolerance towards collagen V or antigenic fragments thereof such that the immune system does not mount a specific anti-collagen V immune response. Relatedly, new methods for diagnosing atherosclerosis involve detecting collagen V in coronary arteries and/or collagen V-specific immunoreactivity.

In a first aspect, the invention is summarized in that methods for modulating an effect of atherosclerosis in a human or non-human subject having, or at risk of developing, the disease include the step of inducing immunological tolerance in the subject. In some embodiments, the tolerance-inducing step includes the steps of administering to the subject collagen V, at least one antigenic fragment thereof or a polynucleotide encoding either of the foregoing, and observing a reduced atherosclerotic burden in the subject.

In some embodiments, a therapeutically effective amount of type V collagen is administered to a patient having atherosclerosis. Type V collagen, or fragment thereof, can be administered, for example, orally, intravenously, intramuscularly, or via any other suitable route.

In a second aspect, the invention is summarized in that methods for determining risk of an individual for developing atherosclerotic cardiovascular disease, such as arteriosclerosis, arteriolosclerosis, and atherosclerosis, includes the step of determining collagen V specific immunoreactivity. In one embodiment, the determining step comprises measuring the amount of collagen V-specific antibodies in a serum sample of an individual. Methods for measuring amounts of specific antibodies are well known in the art and include enzyme-linked immunosorbent assay (ELISA) and flow cytometry. In some embodiments, collagen V fragments are immobilized on a bead and collagen V-specific antibodies binding to the immobilized collagen V are detected through secondary antibody staining. The secondary antibody can be selective for antibody subclasses, e.g., the IgG1-4 subclasses, such that the progression of disease in an individual can be monitored by measuring different collagen V-specific antibody subclass amounts in samples taken at time intervals.

These and other aspects of the invention will be evident upon reference to the following detailed description and attached drawings.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used.

Other objects, advantages and features of the present invention will become apparent from the following specification taken in conjunction with the accompanying drawings.

### BRIEF DESCRIPTION OF THE SEVERAL VIEWS OF THE DRAWINGS

FIG. 1 illustrates trans vivo delayed type hypersensitivity (TV-DTH) responses using peripheral blood mononuclear cells (PBMCs) from individuals having coronary artery disease and from healthy, age-matched controls.

FIG. 2 illustrates TV-DTH responses in the presence of neutralizing antibodies using cells from individuals with coronary artery disease and from healthy controls. PBMCs from CAD patients that had exhibited swelling responses of at least 30×10⁻⁴ inches were coinjected into mouse footpads with 5 μg of neutralizing antibodies to human IFN-γ, TNF-α (both from BD Biosciences, San Jose, Calif.), IL-1β (eBiosciences, San Diego, Calif.), or IL-17 (R&D Systems, Minneapolis, Minn.).

FIG. 3 illustrates TV-DTH responses using peripheral blood mononuclear cells from an individual with coronary artery disease, either undepleted or depleted of CD14⁺ monocytes. Sham depleted cells were run through an immunomagnetic bead column but were not depleted of any cells.

FIG. 4 illustrates TV-DTH responses by splenocytes from ApoE−/− and C57BL/6J control mice.

FIG. 5 illustrates TV-DTH responses by splenocytes from ApoE−/− mice to collagen V alone, or in the presence of neutralizing antibodies.

FIG. 6A-B illustrate TV-DTH responses to collagen V (FIG. 6A) or tetanus toxoid/diphtheria-tetanus toxoids pediatric vaccine (MDT, FIG. 6B) by ApoE−/− mice splenocytes either undepleted or depleted of various cells.

FIG. 7 illustrates serum levels of antibodies specific for col(V) in ApoE−/− and C57BL/6J control mice.

FIG. 8 illustrates the percent lumen occlusion calculated from aortic root section of col(V) sensitized and untreated ApoE−/− mice.

### DETAILED DESCRIPTION OF PREFERRED EMBODIMENTS

From new evidence of an inflammatory autoimmune response to collagen V in human subjects having coronary artery disease, the inventors appreciated that it would be possible to modulate atherosclerosis, to reduce the burden, symptoms or effects of the disease, or to prevent occurrence of the disease in the first instance. The methods comprise administering to a subject a tolerance-inducing antigenic agent in an amount effective to induce tolerance to collagen V in the subject. Where the goal of the method is to treat or modulate atherosclerosis or the effects thereof, the subject is a human or non-human animal having the disease. Where the goal is to prevent occurrence of the disease, the subject is a healthy human or non-human animal, where healthy is defined relative to this disease.

Suitable forms of tolerance-inducing antigenic agent can harbor a continuous- or discontinuous epitope and can include the collagen V protein (GenBank/eMBL Accession No. M76729 and J04478) and individual alpha chains of collagen V, as well as antigenic peptides of collagen V, another antigenic fragment of any of the foregoing, or a polynucleotide encoding any of the foregoing, including the collagen V gene or a portion thereof (GenBank Accession No. NM_{—}000093). Preferably, the tolerance-inducing antigen is allogeneic, but can be xenogeneic, corresponding to a collagen V protein, a collagen V chain, an antigenic peptide or another antigenic fragment of any of the foregoing from other species. Collagen V is highly conserved among human and non-human species, including mammalian and rodent species. Suitable antigens, referred to collectively herein as col(V), can be obtained from bacteria, plants or animals (prokaryotes and eukaryotes) that produce col(V), or fragments thereof, either natively or after being engineered to do so, or can be synthesized de novo.

Collagen V and fragments thereof can be obtained from a variety of commercial (e.g., Collaborative Biomedical Products/Becton, Dickinson and Company, Franklin Lakes, N.J. USA) and non-commercial sources and can be further purified or modified, if required.

The col(V) is administered to an individual in a manner that induces tolerance towards collagen V. The administration can be parenteral (e.g. injection, infusion), topical (e.g. inhalation, intranasal administration), enteral (e.g. oral-, rectal administration) or in any other manner suitable to induce tolerance. Alternatively, a polynucleotide encoding the tolerance-inducing antigen can be introduced to an individual such as to express the protein or peptide. The composition inducing tolerance to collagen V can be administered once or a plurality of times. The skilled person is familiar with pharmaceutically acceptable formulations suitable for administering the tolerance-inducing antigen to a subject.

Collagen V polynucleotide and polypeptide sequences are known to the skilled person and are available in public databases. As used herein, a “polypeptide” refers to a molecule comprising a sequence of amino acids of any length and with or without post-translational modifications, such as glycosilation, both naturally occurring and artificially created. Polypeptides used in the described methods can span all or portions of a protein. In some embodiments, certain amino acids are substituted in the polypeptide compared to the respective naturally-occurring protein, such that the polypeptide has about 70% to about 100% sequence identity to the naturally occurring protein. Methods for effectuating amino acid substitutions are well known in the art and include site directed mutagenesis.

Polypeptides used in the described methods can also be modified to enhance stability or bioavailability using methods well known in the art, such as using alternative bases or peptide backbone linkages. Further, polypeptides can include leader sequences, tags, and/or linkers to direct polynucleotide transfer, facilitate synthesis, identification, and isolation of the polypeptide. Polypeptides used in the described methods can also be fusion polypeptides comprising amino acid sequences from more than one naturally occurring protein or variants thereof.

In some embodiments, a polynucleotide encoding the tolerance-inducing col(V) antigen can be introduced to an individual such as to express the protein or peptide. As used herein, a “polynucleotide” refers to a molecule comprising a sequence of nucleotides of any length and with or without modifications. Polynucleotides used in the described methods can be obtained from a variety of sources well known in the art, such as through purification and/or amplification from cells or tissues or by artificial synthesis. Also, polynucleotides used in the described methods can comprise naturally occurring sequences or variants thereof, as well as artificial sequences and hybrids of naturally occurring and hybrid sequences.

As used herein, the term “tolerance” refers to an unresponsiveness of an individuals' immune system to a specific antigen. Tolerance can be induced by administering an antigen to an individual through various routes, including administering the antigen intravenously, intramuscularly, subcutaneously, intradermally, orally, and nasally. The antigen can be administered, for example, in form of a polypeptide, a precursor- or pre-polypeptide, a polynucleotide encoding the antigen. Specifically contemplated is the induction of tolerance that results in prevention, suppression, or alleviation of cardiovascular disease, such as atherosclerosis.

Specifically contemplated for use with the disclosed methods are the collagen V alpha chain or fragments thereof. In certain embodiments, col(V) administered to an individual can comprise the collagen V homotrimer or fragments thereof. In some embodiments, two or more collagen V fragments are administered to an individual, either concurrently as fusion protein or hybrid polynucleotide or sequentially.

The following examples are provided as further non-limiting illustrations of methods for preventing and ameliorating atherosclerosis.

### EXAMPLES

### Example 1

### Cells from Patients Having Coronary Artery Disease (CAD) are Immunoreactive to Col(V)

Blood samples from twenty patients having end-stage coronary artery disease (CAD) awaiting coronary artery bypass grafting (CABG) and from twenty healthy, age-matched volunteers as controls were tested for cellular anti-collagen V immunoreactivity using the trans vivo delayed type hypersensitivity (TV-DTH) assay. Subject consent was obtained using human subjects committee-approved, written, informed consent procedures at the University of Wisconsin Hospital and Clinics.

Blood samples were collected and processed as described by Rodriguez et al., Am. J. Transplant 4:537-543 (2004), incorporated herein by reference as if set forth in its entirety. Peripheral blood mononuclear cells (PBMCs) were tested within 24 h of isolation.

Trans vivo delayed type hypersensitivity (TV-DTH) was performed by co-transferring human PBMCs and the antigen under investigation into the footpads of CB-17 SCID mice (purchased from Harlan Sprague-Dawley Inc. or bred locally) as described by VanBuskirk, et al. J. Clin. Invest. 106:145-155 (2000); Carrodeguas, et al. Human Immunology 60:640-651 (1999); and Burlingham, et al. Measuring Immunity: basic science and clinical practice. M T. Lotze and A. W. Thomson, editors. Elsevier. San Diego, Calif., USA/London, United Kingdom, 407-418 (2005), each incorporated herein by reference as if set forth in its entirety. All animals were housed and treated in accordance with NIH guidelines, using protocols approved by the Research Animal Resources Center of the University of Wisconsin.

Antigens were injected into the murine footpad (5 μg per injection). Purified collagen V from human placenta was prepared as described by Yasufuku et al., Am. J. Resp. Cell Mol. Biol. 25:26-34 (2001), incorporated herein by reference as if set forth in its entirety. Bovine collagen II (BD Biosciences) was a control antigen not found in the cardiovascular system. Inactivated Epstein-Barr virus (EBV) (Viral Antigens Inc., Saco, Me.) was a control antigen for adequate immune memory function. In some experiments, neutralizing antibody against human IFN-gamma, IL-17, IL-22, IL-1beta (eBiosciences), and TNF-alpha were coinjected with the antigen.

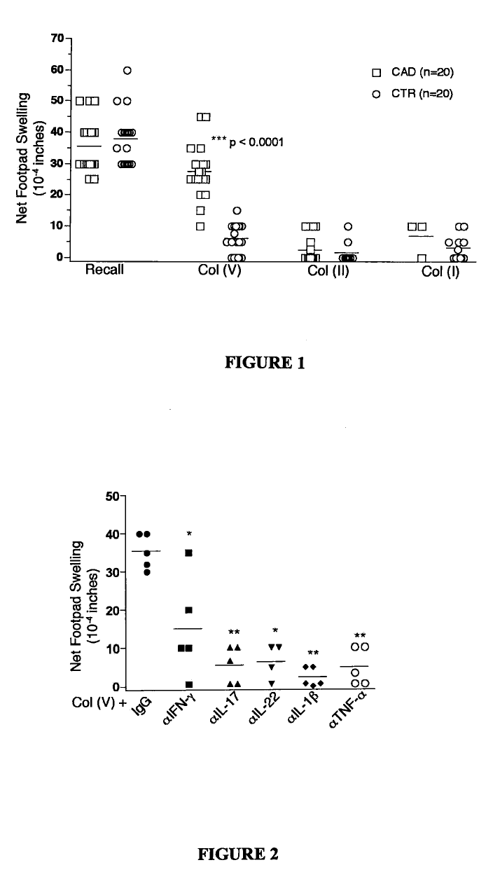

DTH was assessed by measuring the footpad thickness prior to injection and 24 h after injection. Immunoreactivity was quantified as the difference in footpad thickness (in 10⁻⁴ inches) before and after injection. Antigen-specific response was calculated by subtracting from the measured post-injection footpad swelling the swelling observed after control injections of PBMCs with buffer alone. Measurements from duplicate tests were averaged and are shown as individual data points (FIG. 1). The horizontal bars indicate the mean of each group. The Wilcoxon rank-sum test was used for statistical analysis.

Cells from CAD patients and normal healthy controls exhibited similar cellular immune responses to the EBV control antigen. Cells from CAD patients, but not from healthy controls, exhibited a statistically significant cellular immune response to col(V) (P<0.001). Neither cells from CAD patients nor from controls mounted an immune response to collagen II. Preliminary analysis revealed no statistically significant difference in the level of collagen V-specific antibodies in serum samples from human CAD patients compared to serum samples from age-matched control individuals. The result might be attributed to the fact that some of the individuals in the control group, while asymptomatic, were afflicted with atherosclerosis.

The cellular immune response of CAD patient PBMCs was directed against the α1(V) chain. Injection of 0.2-25 μg of either α1(V) chains, α2(V) chains, or α1(V)₂α2(V) heterotrimers resulted in cellular immune response of CAD patients' PBMCs in TV-DTH assays to the α1(V) chain, but little-to-no response to the α2(V) chain. The TV-DTH response from control patient PBMCs remained low against both α1(V)₂α2(V) heterotrimers and the separate col(V) chains, even at high concentrations.

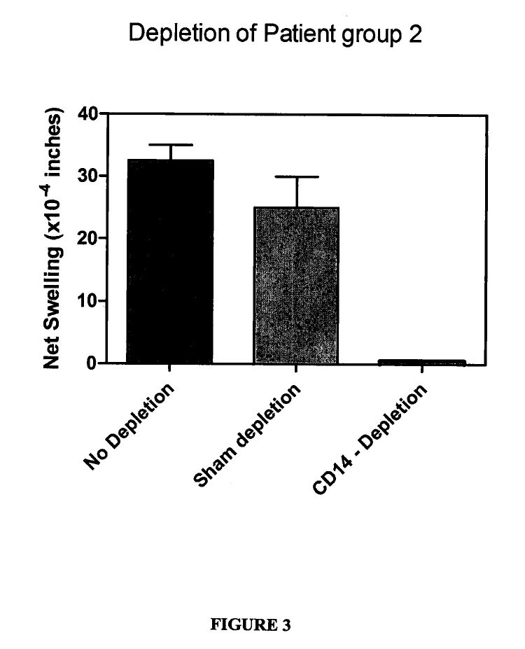

The immunoreactivity of CAD patient PBMCs against collagen V depends upon IL-17, a cytokine released by Th-17 CD4+ T memory cells, and to some varying extent upon IFN-γ, a cytokine released by Th-1 cells. Collagen V-specific TV-DTH responses were consistently blocked by IL-17-neutralizing antibodies (FIG. 2; one star indicates a p value equal to or less than 0.05; two stars indicate a p value equal to or less than 0.01.). The effect of coinjection of IFN-γ-neutralizing antibodies varied between CAD PBMCs from different patients (FIG. 2). Consistent with a role of the Th-17 pathway, neutralizing antibodies to IL-17-induced monokines TNF-α and IL-1β consistently blocked the response, as did neutralizing antibodies to IL-22, another cytokine secreted by Th-17 CD4+ T cells. Immunoreactivity was also abolished by depleting CD14⁺ cells, such as monocytes, from the PBMCs using immunomagnetic beads (MACs beads; Miltenyi Biotec, Bergisch Gladbach, Germany) before conducting the TV-DTH assays (FIG. 3). This result is consistent with a role of the Th-17 pathway in the CAD anti-collagen V response.

### Example 2

### Cellular Immunity to Collagen V in ApoE−/− Mice on a High Fat Diet

The inventors used a relevant murine model system to explore the association of collagen V immunoreactivity with atherosclerosis observed in the human subjects in Example 1. Although rodents typically have high HDL and low LDL/cholesterol levels and are resistant to atherosclerosis, mice deficient in the plasma cholesterol-clearing apolipoprotein E (ApoE−/−) have high plasma cholesterol levels and spontaneously develop atherosclerotic lesions, which dramatically increase on high cholesterol/high fat diets. This widely accepted murine model has been demonstrated to be predictive of human atherosclerosis (Lichtman et al., Am. J. Pathol. 149:351-357 (1996); Zhang et al., Science 258:468-471 (1992); Plump et al. Cell 71:343-353 (1992), each incorporated herein by reference as if set forth in its entirety). As in human atherosclerosis, early and advanced lesions in ApoE−/− mice are heavily infiltrated by activated CD4⁺ T cells and macrophages that contribute to plaque formation.

ApoE−/− mice (strain B6.129P2-Apoe^{tmiUnc}/J) and C57BL/6J (B6) control mice having the same genetic background were obtained from Jackson Laboratory (The Jackson Laboratory, Bar Harbor, Me.). At 8 weeks of age, mice were shifted from a normal diet (Harlan Teklad Rodent Diet 8604, Harlan Laboratories, Inc., Madison, Wis.) to a high-fat “Western” diet (TD.88137 Adjusted Calories Diet, 42% from fat, Harlan Teklad, Madison, Wis.). After 15 weeks on the high-fat diet, mice were sacrificed and spleens were collected for TV-DTH assays.

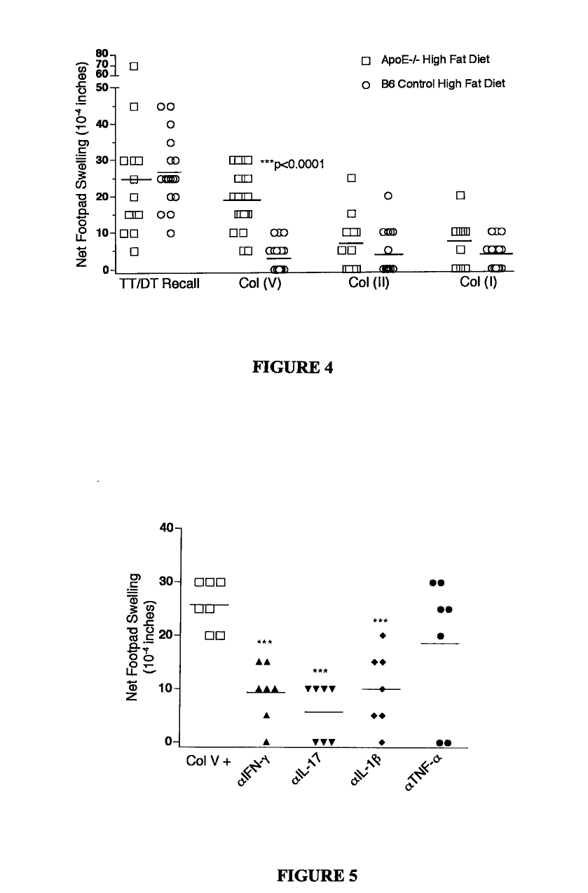

As in Example 1, immunoreactivities against collagen V (Col V), I (Col I), II (Col II), or IV (Col IV) or tetanus toxoid/diphtheria-tetanus toxoids pediatric vaccine (TT/DT) (Aventis-Pasteur Inc., Swiftwater, Pa.) positive immune memory function control antigens were measured in the footpads of the SCID mice. For the latter, mice were immunized with 1 Lf TT/DT pediatric vaccine subcutaneously in the inguinal pouch two weeks prior to harvesting splenocytes. The respective immunoreactivities were averaged from duplicate tests and are shown as individual data points (FIG. 4). Horizontal bars denote group means.

TV-DTH assays were conducted essentially as described in Example 1, except that 10×10⁶ murine splenocytes were injected into the footpads of naive B6 recipients essentially as described in Molitor-Dart et al., J. Immunol. 179:6749-6761 (2007), incorporated herein by reference as if set forth in its entirety. Bovine collagen II, human collagen I and human collagen IV (BD Biosciences) were used as negative controls for specificity of the anti-collagen V response.

In some experiments neutralizing antibodies against murine IFN-gamma, IL-17, IL-22, IL-1beta (eBiosciences), and TNF-alpha were coinjected with the collagen protein (FIG. 5)

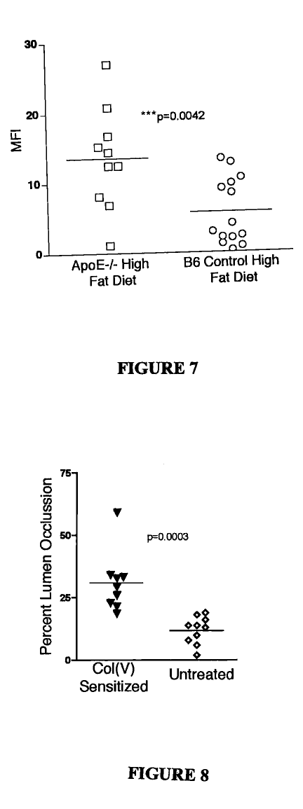

Serum samples were analyzed for col(V)-specific antibodies using a bead assay and flow cytometry (FIG. 7). Streptavidin coated beads (5 μm, binding capacity 10-20 μg)/1×10⁷ beads—Polyscience, Warrington, Pa.) were washed in PBS, suspended in 100 μl of PBS with 40 μg/ml bovine collagen V (ImmuneWorks, Inc., Indianapolis, Ind.), and incubated for 60 minutes at 40° C. The beads were washed in PBS containing 10% fetal calf serum (FCS) and stored at 40° C. until use. For each assay, 1×10⁶ conjugated beads were washed two times in PBS, and incubated in 100 μl PBS and 50 μl of human serum, incubated for 30 minutes at room temperature, and washed in PBS containing 10% FCS. After incubation with anti-human PE-conjugated IgG antibodies (Sigma, St. Louis), the beads were washed and analyzed on a FACSCalibur cytofluorograph (BD Biosciences, Mountain View, Calif.). Collagen V-conjugated beads incubated with biotinylated rabbit anti-human col(V) antibody served as positive controls.

Spleen cells from ApoE−/− mice on the Western diet elicited a specific TV-DTH response to collagen V, while those from C57BL/6J showed no response (FIG. 4). Neither kind of cells responded significantly to collagens I, II or IV (FIG. 4 and data not shown).

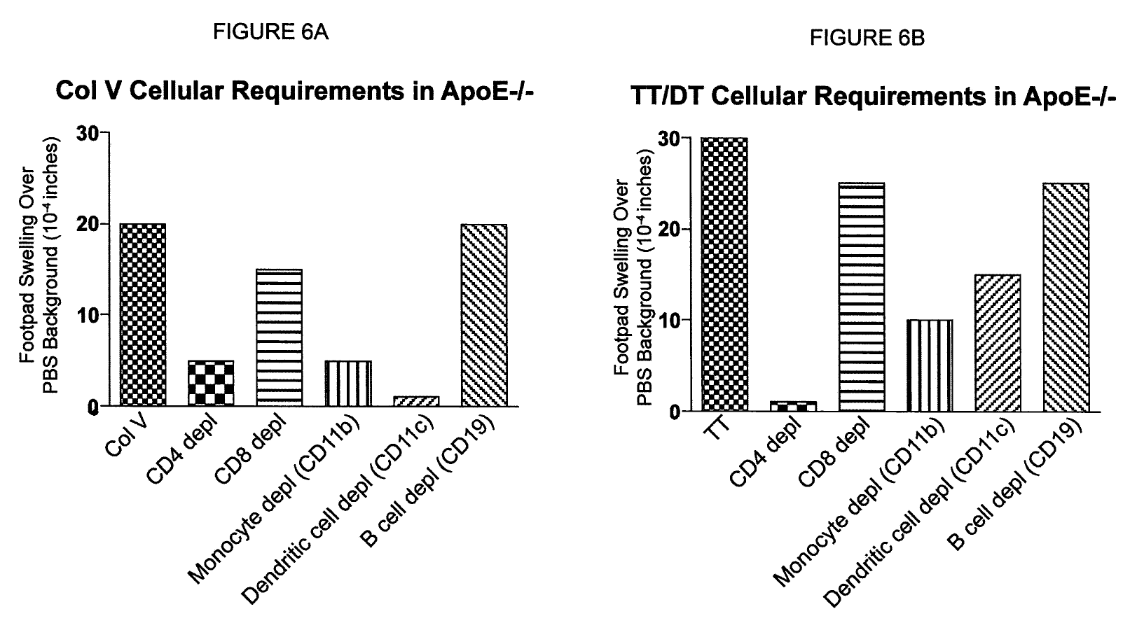

Co-injecting cytokine-neutralizing antibodies and the splenocytes from ApoE−/− mice into murine footpads revealed that like the human anti-collagen response, the murine specific anti-collagen V response appears to depend upon both the Th-17 and Th-1 pathways, since neutralizing antibodies to IL-17, IL-10, and IFN-γ significantly blocked the response (FIG. 5). However, in contrast to the DTH response observed using PBMC from human CAD patients, TNF-α-neutralizing antibodies did not block the cellular anti-collagen V response of ApoE−/− splenocytes (FIG. 5). As a further indication that the Th-17 and Th-1 pathways are involved, no collagen V-specific immunoreactivity was observed when the inventors depleted CD4⁺ cells, CD11b⁺ cells or CD11c⁺ cells (monocytes/macrophages and immature dendritic cells) as in Example 1 (FIG. 6A). Similar results were obtained when assessing response to TT/DT (FIG. 6B), except that when CD11c⁺ cells were depleted a smaller decrease in swelling was observed than was observed in response to collagen V. The inventors observed no effect on collagen V- or TT/DT-specific swelling when CD8⁺ cells or CD19⁺ B cells were depleted (FIG. 6A, B).

ApoE−/− mice on a high fat diet had significantly higher levels of anti-col(V) antibodies compared to B6 control mice on a high fat diet, as determined by serum analysis for collagen V-specific antibodies using a bead assay and flow cytometry (FIG. 7).

### Example 3

### Collagen V Expression in Atherosclerotic Plaques from ApoE−/− Mice

Aortic roots from ApoE−/− mice fed a high fat diet displayed the prototypical lesions, while aortic roots from control B6 mice were free of such lesions. To quantify plaque formation, the hearts were removed and washed in PBS. The heart was then cut transversely and embedded in O.C.T. Compound (Sakura Finetek USA). Serial sections were cut through the entire aortic sinus and sections were stained with hematoxylin and eosin. Percent vessel occlusion was measured using imaging system by measuring the ratio of the vessel intima area (without plaque) to the vessel lumen area (with plaque) and calculated as the mean of 3 section/sample, each at 50 μm apart spanning the aortic sinus.

ApoE−/− mice fed a high fat diet had a significantly higher percentage of vessel occlusion compared to the B6 control mice fed a high fat diet.

For immunohistochemistry, formalin-fixed, paraffin-embedded aortic arches collected from ApoE−/− mice were sectioned at a thickness of 5 microns, deparaffinized, and rehydrated for collagen V staining. Non-specific antibody binding was blocked by 1 hour incubation in 10% bovine serum albumin in PBS, followed by 30 minute incubation in 5% non-fat milk in PBS. Tissue sections were incubated with a rabbit anti-human collagen V primary antibody (LifeSpan Biosciences, Inc., Seattle, Wash.) for 1 hour at room temperature. Endogenous peroxidase activity was quenched with hydrogen peroxide. Primary antibody binding was then detected with Rat on Mouse HRP-polymer kit (Biocare Medical LLC, Concord, Calif.). The signal was visualized using DAB chromogen (Dako) followed by counterstaining with hematoxylin for contrast.

Col(V) is expressed in atherosclerotic lesions of ApoE−/− mice on a high fat diet Collage V was strongly expressed in the intimal and medial layer regions of advanced lesions from ApoE−/− aortae, along with strong expression in some areas of the adventia, whereas B6 control aorta lacked detectable collagen V expression in the intimal and medial layers.

### Example 4

### Collagen V Injection Exacerbates Atherosclerotic Burden

To determine the role of collagen V autoimmunity in atherosclerosis, ApoE−/− mice maintained on regular chow were treated with several intravenous (i.v.) high doses of collagen V to induce sensitization (50 μg collagen per injection every two weeks, eight injections total). After 15 weeks, splenocytes and aortic roots were collected from ApoE−/− mice that received intraveneous collagen V injections and ApoE−/− control mice (all maintained on regular chow) and analyzed for collagen V reactivity and disease pathology. Collagen V injections exacerbated the level of atherosclerotic burden in aortic roots. Aortic sinuses of ApoE−/− mice that were injected with collagen V had an increased plaque burden, compared to aortic sinuses from untreated ApoE−/− mice. Collagen V-injected ApoE−/− mice also had a significant increase in percent vessel occlusion due to the increased plaque burden, compared to untreated ApoE−/− mice (FIG. 8). Further, collagen V-injected ApoE−/− mice had significantly higher titers of anti-collagen V antibodies in their serum than untreated ApoE−/− control mice. Furthermore, collagen V-injected ApoE−/− mice had significantly greater TV-DTH swelling responses to collagen V.

### Example 5

### Prophetic: Reduction of Atherosclerosis or Effects Thereof on Subject Having Atherosclerosis

Collagen V, a collagen V chain, or a peptide or fragment of any of the foregoing (collectively, “col(V)”), or a polynucleotide encoding any of the foregoing is administered to a human or non-human subject having atherosclerosis in a pharmaceutically acceptable carrier such as physiological saline (see, e.g., Yasufuku et al, Am. J. Respir. Cell Mol. Biol. 25:26-34 2001)) or phosphate buffered saline through an acceptable delivery route (e.g., intra-nasal, oral, or the like) (see, e.g., Carbone et al, Arthritis Rheum. 50:2713-2715 (2004), incorporated herein by reference in its entirety as if set forth herein). The aforementioned tolerance inducing agent is administered in an amount sufficient to induce immunological tolerance to collagen V in the subject. The status of the subject as having atherosclerosis can be assessed by conventional methods available to physicians. Tolerance to collagen V is monitored by testing PBMCs in a TV-DTH assay essentially as described in Example 1.

It is observed that the subject's atherosclerosis is reduced, does not progress as would be expected without this treatment or does not exhibit symptoms having the same magnitude or duration as before the treatment.

### Example 6

### Prophetic: Prevention of Atherosclerosis in a Subject at Risk for Atherosclerosis

Collagen V, a collagen V chain, or a peptide or fragment of any of the foregoing (collectively referred to as “col(V)”) is administered in a pharmaceutically acceptable carrier (e.g., a buffer) via an acceptable delivery route (e.g., parenterally, enterally, or topically, especially by intra-nasal, oral, or similar delivery route) to a human or non-human subject at risk of developing atherosclerosis in an amount sufficient to induce immunological tolerance to collagen V in the subject. The at-risk nature of the subject can be assessed using conventional measures, such as elevated LDL concentration in peripheral blood.

Tolerance to collagen V is monitored by testing PBMCs in a TV-DTH assay essentially as described in Example 1, or via a suitable ELISPOT assay. It is observed that the subject does not develop atherosclerosis at a rate that would be expected in the population of subjects sharing the same level of risk.

### Example 7

### Prophetic: Anti-Collagen V DTH as Biomarker of Atherosclerosis

Immune system cells (such as PBMCs) from a subject are tested for immunoreactivity to collagen V using a TV-DTH assay, essentially as described in Example 1, or a suitable ELISPOT assay. A subject's risk for developing atherosclerosis is evaluated whereby evidence of immunoreactivity (i.e., a lack of tolerance to collagen V) is associated with a higher risk than would be expected in a subject from whose PBMCs immunoreactivity is not observed.

We claim:

1. A method for reducing atherosclerosis or an effect thereof in an individual having atherosclerosis, the method comprising the steps of:
inducing immunological tolerance to collagen V in the individual; and
observing reduced atherosclerosis or a reduced effect thereof in the individual.

2. The method of claim 1, wherein the inducing step comprises the steps of administering to the individual an amount effective to induce tolerance of an agent selected from the group consisting of collagen V, a collagen V chain, an antigenic peptide, an antigenic fragment of any of the foregoing and a polynucleotide encoding any of the foregoing.

3. The method of claim 2, wherein the agent comprises a human collagen V gene.

4. The method of claim 2, wherein the agent is synthesized de novo.

5. The method of claim 2, wherein the agent is administered parenterally, enterally, or topically.

6. The method of claim 5, wherein the composition is administered intranasally.

7. The method of claim 1, wherein the composition is administered more than once.

8. A method for assessing a risk of an individual to develop atherosclerosis, the method comprising the steps of:
ascertaining whether the individual is immunoreactive to collagen V; and
assessing the risk of the individual, whereby immunoreactivity is associated with a higher risk than would be expected in an individual from whom immunoreactivity is not observed.

9. The method of claim 8, wherein the ascertaining step comprises measuring the amount of collagen V-specific antibodies.

10. The method of claim 9, wherein the measuring step comprises the steps of:
providing a blood sample of the individual; and
contacting the sample with collagen V or a collagen V fragment.

11. The method of claim 10, wherein the collagen V or collagen V fragment is immobilized on a bead.

12. The method of claim 8, wherein the ascertaining step comprises measuring the amount of collagen V-specific lymphocytes.

13. The method of claim 13, wherein the measuring step comprises evaluating peripheral blood mononuclear cells in a delayed-type hypersensitivity assay.

14. The method of claim 8, wherein the delayed type hypersensitivity assay is selected from the group consisting of a trans vivo delayed type hypersensitivity assay and an ELISPOT assay.
